(19) United States
(12) Patent Application Publication
(10) Pub. No.: US 2004/0203054 A1
(43) Pub. Date: Oct. 14, 2004

(54) Use of SLIM3 for binding to molecules

(76) Inventors: Roland Schule, Weisweil (DE); Judith Muller, Freiburg (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/839,281

(22) Filed: May 6, 2004

Related U.S. Application Data
(62) Division of application No. 09/909,762, filed on Jul. 23, 2001.
Application No. 09/909,762 is a division of application No. 09/428,647, filed on Oct. 28, 1999.

(30) Foreign Application Priority Data
May 21, 1999 (EP) 99250161.9

(51) Int. Cl. C12Q 1/68; A61K 38/17

(52) U.S. Cl. 435/6; 514/12

(57) ABSTRACT

The invention relates to the use of SLIM3 for binding to an androgen receptor or estrogen receptor β. The invention includes the use of a process for identifying ligands, which affect the interaction of SLIM3 and an androgen receptor or estrogen receptor β, so that the transcription, which is controlled by the receptor, is increased or decreased. Ligands, which are antagonists or agonists, are the preferred target of the identification.

This application claims the priority of European Application No. 99 250 161.9, filed May 21, 1999, which is hereby incorporated by reference.

The invention relates to the use of the protein SLIM3 for binding to receptors, in addition a process for screening ligands, which bind to SLIM3, i.a.

### Prior Art

The protein SLIM3 was first described by M. J. MORGAN and A. J. A. MADGWICK (1996) Biochem. Biophys., Res. Commun., Vol. 225, pp. 632-638. SLIM3 has 279 amino acids.

SLIM3 is strongly expressed in the myocardium and weakly expressed in the placenta, in skeletal muscles, in the prostate, in the testes, in the intestine and in the ovaries. The protein SLIM3 could not be detected in the brain, the lung, the liver, the kidneys, the pancreas, the spleen, the thymus gland and in leukocytes. (M. GENINI et al. (1997) DNA Cell Biol., Vol. 16, pp. 433-442). Even though expression was noted, the physiological or pathophysiological function of SLIM3 was still not described. In this respect, the gene family of the LIM proteins, to which SLIM3 belongs, is known to have domains that bind to other proteins.

The nuclear receptor AR (androgen receptor) and its function is described in A. C. B. CATO et al. (1998) The Androgen Receptor as Mediator of Gene Expression and Signal Transduction Pathways, TEM, Vol. 9, pp. 150-154, and Z. X. ZHOU et al. (1994) The Androgen Receptor: An Overview, Recent Prog Horm Res, Vol. 49, pp. 249-274 and E. M. WILSON et al. (1991) Molecular Analysis of the Androgen Receptor, Ann N Y Sci, Vol. 637, pp. 56-63.

The nuclear receptor ERβ and its function (estrogen receptor β) is described by V. GIGUERE et al. (1998) Estrogen Receptor Beta: Reevaluation of Estrogen and Antiestrogen Signaling; Steroids Vol. 63, pp. 335-339 and G. G. KUIPER et al. (1997) The Novel Estrogen Receptor Subtype: Potential Role in the Cell- and Promoter-Specific Actions of Estrogens and Antiestrogens, FEBS Lett., Vol. 410: pp. 87-90.

The above-mentioned nuclear receptors bind to promoters, which regulate genes that are responsive in the natural environment (responsive genes), such as probasin, MMTV (mouse mammary tumor virus), C3 and retinoic acid receptor alpha 1 (M. G. PARKER et al. (1987) Identification of Androgen Response Elements in Mouse Mammary Tumor Virus and the Rat C3 Gene. J. Ce. Biochem Vol. 35, pp. 285-292 and F. CLAESSENS et al. (1996) The Androgen-Specific Probasin Response Element 2 Interacts Differentially with Androgen and Glucocorticoid Receptors, J. Biol Chem., Vol. 271, pp. 19013-19016 and A. ZOU et al. (1999) Estrogen Receptor Beta Activates the Human Retinoic Acid Receptor Alpha-1 Promoter in Response to Tamoxifen and Other Estrogen Receptor Antagonists, But Not In Response to Estrogen, Mol Endocrinol Vol 13, pp. 418-430) (J. SAMBROOK et al. (1989) Molecular Cloning. Cold Spring Harbour Laboratory Press, New York).

### Object and Achievement

An object of the invention are methods and compositions comprising SLIM3 for regulating and interacting specifically with proteins. An object of the invention is to identify ligands that affect the interaction between SLIM3 and the proteins to which it specifically binds.

The objects can be achieved by the use of SLIM3 (=protein) or a modification thereof for binding to at least one of the nuclear receptors

(i) AR (androgen receptor) (=protein) or a modification thereof, and

(ii) ERβ (estrogen receptor β) (=protein) or a modification thereof,

whereby the modification consists in that up to ten amino acids in the respective modified protein are deleted, substituted or inserted compared to the corresponding natural protein,

without in this case the function of the respective modified protein being significantly affected in comparison to the corresponding natural protein.

The invention also comprises the use of the amino acid sequence for SLIM3 (=protein),

which is coded by a cDNA, for binding to at least one amino acid sequence for the nuclear receptors (=protein)

(i) AR (androgen receptor), which is coded by a cDNA, and

(ii) ERβ (estrogen receptor β), which is coded by a cDNA, whereby optionally one or more cDNAs,

which are coded for SLIM3, AR or ERβ, are modified and in this case are at least about 85% homologous to the cDNA sequence,

which codes for the natural SLIM3, AR or ERβ, without the function of the respective, expressed protein being significantly affected by the modification of the cDNAs in comparison to the natural protein.

The use of SLIM3 according to the invention is preferred, whereby nuclear receptors AR and ERβ control the transcription of the cells.

More preferred is the use of SLIM3 according to the invention as a functional co-activator for nuclear receptors AR and ERβ.

Most preferred is the use of SLIM3 according to the invention for increasing the transcription of nuclear receptors AR and ERβ. The invention preferably relates to human SLIM3, human AR, and human ERβ.

SLIM3 was described in M. J. MORGAN and A. J. A. MADGWICK (1996) Biochem. Biophys. Res. Commun., Vol. 225, pp 632-638. Androgen—receptor described in A. C. B. CATO et al. (1998). The androgen/receptor as mediator of gene expression and signal transduction pathways, TEM, Vol. 9, pp 150-154. Estrogen receptor β is described in V. GIGUERE et al. (1998) Estrogen receptor beta: re-evaluation of estrogen and anti-estrogen signaling; Steroids, Vol. 63, pp 335-339.

### Definitions

Modification of SLIM3 and nuclear receptors AR and ERβ with allelic and post-translational modifications:

### Allelic Modifications

Most deletions, insertions and substitutions do not appear to result in any drastic change in the characteristics of proteins SLIM3, AR and ERβ. The function of the altered protein can be compared to the function of the known protein. As a standard for SLIM3, the protein is used according to the publication by K. K. CHAN et al. (1998) Molecular Cloning and Characterization of FHL2, A Novel LIM Domain Protein Preferentially Expressed in Human Heart, Gene, Vol. 210, pp. 345-350 and M. GENINI et al. (1997) Substractive Cloning and Characterization of DRAL, A Novel LIM—Domain Protein Down-Regulated in Rhabdomyosarcoma, DNA Cell Biol., Vol. 16, pp. 433-442 and M. J. MORGAN and A. J. A. MADGWICK (1996) Biochem. Biophys. Res. Commun., Vol. 225, pp. 632-638 (=natural SLIM3). For nuclear receptors (=natural AR and ERβ), the proteins (=natural AR and ERβ), which are described in the publications via AR and ER, are used as standards: for AR: A. C. B. CATO et al. (1998) The Androgen Receptor as Mediator of Gene Expression and Signal Transduction Pathways, TEM, Vol. 9, pp. 150-154, and Z. X. ZHOU, et al. (1994) The Androgen Receptor: An Overview; Recent Prog Horm Res, Vol. 49, pp. 249-274 and E. M. WILSON et al. (1991) Molecular Analysis of the Androgen Receptor; Ann N Y Sci, Vol. 637, pp. 56-63; in addition for ERβ: V. GIGUERE et al. (1998) Estrogen Receptor Beta: Re-Evaluation of Estrogen and Antiestrogen Signaling; Steroids Vol. 63, pp. 335-339 and G. G. KUIPER et al. (1997) The Novel Estrogen Receptor Subtype: Potential Role in the Cell- and Promoter-Specific Actions of Estrogens and Antiestrogens, FEBS Lett, Vol. 410: pp 87-90.

Mutations are defined by the homology (similarity) of two proteins or genes that are being compared.

Amino acids can be substituted, as shown in Table 1, without, in this case, significantly affecting the function of the respective protein. In each individual case, it can be decided by the activity test what effect the change on the function of the protein has.

The functions or the immunological identity are significantly changed when substituents are selected that are less conservative than the amino acids that are shown in Table 1 in the case of substitution. Such significant changes can be achieved by substitutions with amino acids, which are distinguished more in their structure and in the functional groups. Significant changes have the effect that the three-dimensional structure is altered and/or that, for example, the pleated-sheet structure or the helical structure is affected. Interactions of the charges and the hydrophobic chains can also be noted in the changes.

By the term “without significantly affecting,” it is meant that the protein still possesses its normal biological activity as measured in the publications described above. Such activity can be less than 100% as compared to a protein having its naturally-occurring sequence, preferably it possesses 50% or more, more preferably 60% or more, 75% or more, and most preferably 80% or more, of its normal activity.

Methods and agents identified in accordance with the present invention can be useful in various ways, e.g., as research tools, to increase the transcription of genes activated by the AR and ERβ receptors, such as in conditions of steroid (androgens, estrogens) deficiency.

TABLE 1
Commonly Used Substitution of Amino Acids in a Protein

| Original | Substitution Carried Out, |
|---|---|
| Amino Acid | For Example |
| Ala | Gly, Ser |
| Arg | Lys, |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

Such analyses can be readily achieved via substitutions. In this case, in one position an amino acid is exchanged for preferably alanine or another amino acid. After the synthesis of the modified protein, the function of the altered protein is measured, by the binding ability and the ability to be a co-factor being tested in SLIM3. In the case of the receptors, other tests are useful that measure the function to bind SLIM3 and that measure the function to affect the transcription. The functions are described in the respective bibliographic references concerning AR and ERβ.

Homology on the Level of the Amino Acid Sequences:

The term homology comprises similar amino acids (for example as expressed in Table 1) and gaps in the sequences of amino acids (homology=similarity). The proteins in the application have amino acid sequences that have a homology of at least 90%, preferably 95%, more preferably 98% and most preferably 99% of the described structures (SLIM3 and the two nuclear receptors). Homologous amino acids also include those described by Dayhoff in the “Atlas of Protein Sequence and Structure,” 5, 1978 and Argos et al., EMBO J., 8:779-785, 1989.

Homology on the Level of DNA:

Homology in the case of nucleotide sequences means that two polynucleotide or corresponding sequences, if they are optimally placed with respect to one another and optimally engage in one another, are identical. The homology can also exist only partially, however. Thus, in the case of SLIM3 and the receptors, a homology of at least 85%, preferably 92%, more preferably 98% and most preferably 99% is to be present. The homology is determined via the hybridization of two nucleotide strands, as is described in J. COOMBS (1994) Dictionary of Biotechnology, Stockton Press, New York. The homology can also be measured as it is described in R. KNIPPERS, Molekulare Genetik [Molecular Genetics], 1982, Third Edition, Georg Thieme Verlag Stuttgart, New York.

Derivatives of proteins, such as SLIM3, AR, or ERβ, can be coded for by nucleic acids selected on the basis of nucleic acid hybridization. The ability of two single-stranded nucleic acid preparations to hybridize together is a measure of their nucleotide sequence complementarity, e.g., base-pairing between nucleotides, such as A-T, G-C, etc.

Hybridization conditions can be chosen to select nucleic acids which have a desired amount of nucleotide complementarity with the naturally-occurring nucleotide sequences. A nucleic acid capable of hybridizing to such sequence, preferably, possesses, e.g., about 85%, more preferably, 90%, 92%, and even more preferably, 95%, 97%, or 100% complementarity, between the sequences. The present invention particularly relates to nucleic acid sequences which hybridize to the nucleotide sequence set forth in FIGS. 1-6 under low or high stringency conditions.

Nucleic acids which hybridize to known sequences of SLIM2, AR, or ERβ, can be selected in various ways. For instance, blots (i.e., matrices containing nucleic acid), chip arrays, and other matrices comprising nucleic acids of interest, can be incubated in a prehybridization solution (6× SSC, 0.5% SDS, 100 μg/ml denatured salmon sperm DNA, 5× Denhardt's solution, and 50% formamide), at 30° C., overnight, and then hybridized with a detectable oligonucleotides probe, (see below) in a hybridization solution (e.g., 6× SSC, 0.5% SDS, 100 μg/ml denatured salmon sperm DNA and 50% formamide), at 42° C., overnight in accordance with known procedures. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity. Other non-limiting examples of high stringency conditions includes a final wash at 65° C. in aqueous buffer containing 30 mM NaCl and 0.5% SDS. Another example of high stringent conditions is hybridization in 7% SDS, 0.5 M NaPO4, pH 7, 1 mM EDTA at 50° C., e.g., overnight, followed by one or more washes with a 1% SDS solution at 42° C.

Whereas high stringency washes can allow for less than 5% mismatch, relaxed or low stringency wash conditions (e.g., wash twice in 0.2% SSC and 0.5% SDS for 30 min at 37° C.) can permit up to 20% mismatch. Another non-limiting example of low stringency conditions includes a final wash at 42° C. in a buffer containing 30 mM NaCl and 0.5% SDS. Washing and hybridization can also be performed as described in Sambrook et al., Molecular Cloning, 1989, Chapter 9.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6log10[Na+]+0.41(%GC)−600/N where [Na+] is the molar concentration of sodium ions, %GC is the percentage of GC base pairs in the probe, and N is the length. Hybridization can be carried out at several degrees below this temperature to ensure that the probe and target can hybridize. Mismatches can be allowed for by lowering the temperature even further.

### Posttranslational Modifications

The above-mentioned posttranslational modifications are defined as changes that occur during or after translation. This includes glycosylation, the structuring of disulfide bridges, the chemical modifications of amino acids, thus, for example, sulfation, which is described in connection with hirudin. (J. W. FENTON (1989) “Thrombin Interactions with Hirudin,” Seminars in Thrombosis and Hemostasis 15: 265-268). Phosphorylation and proteolysis also fall under these modifications.

Glycosylation is a basic function of the endoplasmatic reticulum and/or the Golgi apparatus. The sequence and the branching of the oligosaccharides are formed in the endoplasmatic reticulum and altered in the Golgi apparatus. The oligosaccharides can be N-linked oligosaccharides (asparagine-linked) or O-linked oligosaccharides (serine-, threonine- or hydroxylysine-linked). The form of the glycosylation depends on the cell type that is produced and on the type that originates from the corresponding cell type. The extent and the type of glycosylation can be affected by substances as described in European Publication EP 0 222 313. The variation of the glycosylation can alter the function of the protein.

Proteins frequently form covalent bonds within the chains. These disulfide bridges are produced between two cysteines. In this case, the protein is specifically folded. The disulfide bridges stabilize the three-dimensional structure of the proteins.

SLIM3 modifications and derivatives are determined in that the function according to the examples of the SLIM3 that is described in the literature is compared to the modification.

A SLIM3 derivative which can be used in the present invention can possess one or more of such mentioned activities or measured conventionally or described therein in the examples.

Nuclear receptor AR modifications are determined in that the functions of the DNA bond, conformation, co-factor-bond and the transcriptional properties of nuclear receptor AR that is described in the literature are compared to that of the modifications (A. C. B. CATO et al. (1998) The Androgen Receptor As Mediator of Gene Expression and Signal Transduction Pathways, TEM, Vol. 9, pp. 150-154, and Z. X. ZHOU, et al. (1994) The Androgen Receptor: An Overview. Recent Prog Horm Res, Vol. 49, pp. 249-274 and E. M. WILSON et al. (1991) Molecular Analysis of the Androgen Receptor. Ann N Y Sci, Vol. 637, pp. 56-63 and J. TORCHIA et al. (1998) Co-Activators and Co-Repressors in the Integration of Transcriptional Responses. Curr Opin Cell Biol. Vol. 10, pp. 373-383 and D. J. MANGELSDORF et al. (1995) The Nuclear Receptor Superfamily: The Second Decade, Cell, Vol. 83, pp. 835-839). An AR derivative which can be used in the present invention can possess one or more of such mentioned activities as measured conventionally or described herein in the example.

Any gene which comprises a response element the human AR or ERβ receptors would be useful to determine transcriptional activating activity in accordance with the present invention. For example, the functional co-activating activity of SLIM3 on a human AR or ERβ can be measured by assaying for expression of a gene having an element which is responsive to human AR or ERβ. An example of such genes are described in the examples.

Androgen receptor described in A. C. B. CATO et al. (1998). The androgen receptor as mediator of gene expression and signal transduction pathways, TEM, Vol. 9, pp 150-154. Estrogen receptor β is described in V. GIGUERE et al. (1998). Estrogen receptor beta: re-evaluation of estrogen and anti-estrogen signaling; Steroids, Vol. 63, pp 335-339.

Nuclear receptor ERβ modifications are determined in that the functions of the DNA bond, conformation, co-factor bond and the transcriptional properties of nuclear receptor ERβ that is described in the literature are compared to that of the modifications (V. GIGUERE et al. (1998) Estrogen Receptor Beta: Re-evaluation of Estrogen and Antiestrogen Signaling. Steroids Vol. 63, pp. 335-339 and G. G. KUIPER et al. (1997) The Novel Estrogen Receptor Subtype: Potential Role in the Cell- and Promoter-Specific Actions of Estrogens and Antiestrogens, FEBS Lett, Vol. 410: pp. 87-90 and J. TORCHIA et al. (1998) Co-activators and Co-repressors in the Integration of Transcriptional Responses. Curr Opin Cell Biol. Vol. 10, pp. 373-383 and D. J. MANGELSDORF et al. (1995) The Nuclear Receptor Superfamily: The Second Decade, Cell, Vol. 83, pp. 835-839).

An ERβ derivative which can be used in the present invention can possess one or more of such mentioned activities as measured conventionally or described herein in the examples.

Ligand: The ligands include all natural and synthetically-produced substances that can bind to a receptor and/or to SLIM3. The ligand can also be an antibody that is directed against a receptor and/or SLIM3. Ligands can be agonists or antagonists.

### Screening Process

The invention also comprises a process for identifying (screening) ligands,

which affect the interaction of SLIM3 or modifications thereof and of at least one of nuclear receptors AR or ERβ or modifications thereof,

such that the transcription,

which is controlled by the nuclear receptor,

is increased or reduced in the absence of the ligand,

and is used according to the invention by SLIM3 (=protein) or a modification thereof

for binding to at least one of the nuclear receptors

(i) AR (androgen receptor) (=protein) or a modification thereof and

(ii) ERβ (estrogen receptor β) (=protein) or a modification thereof.

The modifications are defined according to the preceding description.

Preferred is a process according to the invention whereby the ligand is an antagonist or an agonist. In this respect, specific antagonists and agonists can be found that act on individual organs, since SLIM3 is expressed specifically in a few tissues. The ligands that are found can thus be used as medication or as a guide structure for discovering a medication.

Comparable screening methods are described (A. C. B. CATO et al. (1998) The Androgen Receptor as Mediator of Gene Expression and Signal Transduction Pathways, TEM, Vol. 9, pp. 150-154 and G. Zhou et al. (1998) Nuclear Receptors Have Distinct Affinities for Co-activators: Characterization by Fluorescence Resonance Energy Transfer, Mol. Endocrinol. Vol. 12, pp. 1594-1604).

Indicators for transcription are described in J. Sambrook et al. (1989) Molecular Cloning. Cold Spring Harbour Laboratory Press, New York.

Additional GST-protein-interaction tests also showed that SLIM3 interacts with known co-activators TIF2, RIP140 and AIB/ACTR. No interaction between SLIM3 and co-activator SRC1 exists in vitro, however.

Thus, the present invention relates to methods of identifying ligands which regulate the interaction of SLIM3 with various proteins, including nuclear receptors, such as human AR or ERβ.

### EXAMPLES

### Expression of SLIM3 in Various Tissues

Northern Blots and in situ analyses have shown that SLIM3 is expressed during embryonal development from day nine of development. By the twelfth day of embryonal development, SLIM3 is also detected in the visceral and cardiovascular smooth muscles. This specific expression lasts until later in life. No expression could be determined in the following tissues: brain, lung, liver, kidneys, pancreas, spleen, thymus gland and the peripheral blood-leukocytes. The tests were carried out according to M. MOSER et al. (1997) Comparative Analysis of AP-2 Alpha and AP-2 Beta Gene Expression During Mice Embryogenesis. Dev Dyn, Vol. 208, pp. 115-124. Cell cultures and transient transfection tests: 293, CV1 and A10 cells were cultivated in Dulbecco's Modified Eagle's Medium (DMEM), in which 10% or 20% (A10) fetal calf serum was found. HL-1 was cultivated in Ex-cell 320, as is described in W. C. CLAYCOMB et al. (1998) HL-1 Cells: A Cardiac Muscle Cell Line that Contracts and Retains Phenotypic Characteristics of the Adult Cardiomyocyte, Proc Natl Acad Sci USA, Vol. 95, pp. 2979-2984. Transient transfection tests in 293 cells were carried out according to the standard of calcium phosphate co-precipitation technique (Greiner, E. F., J. Kirfel, H. Greschik, U. Dörflinger, P. Becker, A. Mercep, and R. Schüle. 1996. Functional Analysis of Retinoid Z Receptor β, A Brain-Specific Nuclear Orphan Receptor. Proc. Natl. Acad. Sci. USA 93: 10105-10110).

A10 and CV-1 were transfixed, whereby DOTAP was used corresponding to the manufacturer's recommendation of Boehringer Mannheim. HL-1 was transfixed, whereby effects corresponding to the manufacturer's recommendation of quiagen were used. The cells were incubated after 22 hours with or without hormones, and, as recommended by Promega, the luciferase activity was measured in a luminometer ML3000 (Dynatech). The relative light units were balanced against the protein concentration corresponding to the Bradford color test (BioRad). All experiments were repeated five times.

### SLIM3 Specific Interaction with the Ligand-Activated Androgen Receptor In Vivo

To measure the interaction between SLIM3 and the androgen receptor, the liquid β-galactosidase test was used. DNA of the androgen receptor was therefore replaced by a GAL4 DNA binding domain. The chimera protein (AGA) that is obtained, which contains the human androgen receptor-N-terminus and the androgen receptor-domain (LBD) that binds ligands, was used as a target protein. SLIM3, which was fused with the GAL4 activator domain (Slim3-AD), was used as a binding protein. Slim3-AD, which is connected with AGA in a ligand-dependent type, increases the β-galactosidase reporter gene activity in yeast by more than tenfold. See, e.g., U.S. Pat. Nos. 5,283,173, 5,468,614, and 5,667,973.

### SLIM3 Interacts Specifically with Some Nuclear Receptors In Vitro

To measure the interaction between SLIM3 and the nuclear receptors in vitro, the GST-Slim3 fusion protein was immobilized by means of glutathione-sepharose and incubated with several in vitro-translated ³⁵S-methionine-labeled nuclear receptors. The complete human androgen receptor binds to GST-Slim3. This in vitro interaction is not ligand-dependent under standard conditions. The complete human androgen receptor binds specifically to GST-Slim3, but not to the GST protein alone. To safeguard the specificity between the human androgen receptor and SLIM3, another GST-protein-interaction test was carried out (GST-protein-interaction assay). This test is described in E. PFITZNER et al. (1995) Functional Antagonism Between the Retinoid Acid Receptor and the Viral Transactivator BZLF1 is Mediated by Protein-Protein Interactions. Proc Natl Acad Sci USA Vol. 92, pp. 12265-12269. Specific ligand-independent interactions between the GST-SLIM3 fusion protein and the ERβ receptor could be observed. All other tested nuclear receptors, for example progesterone receptors alpha and beta, glucocorticoid receptors and estrogen receptors alpha showed no bond to SLIM3.

Other GST-protein interaction tests also showed that SLIM3 interacts with known co-activators TIF2, RIP140 and AIB/ACTR. There is no interaction between SLIM3 and the co-activator SRC1 in vitro, however. SLIM3 is a functional co-activator:

It was possible to show in experiments that SLIM3 does not bind to DNA. SLIM3 is a co-activator for specific receptors and in specific cell types. Gal-SLIM3, which was found in an expression plasmid, was co-transfixed in various mammal cell lines. In this case, the question was whether Gal-SLIM3 is capable of transactivating a luciferase reporter gene, which is controlled by Gal binding sites, such as, for example, the three Gal4 binding sites upstream from the thymidine kinase promoter [(GAL)3-TKLuC]. Gal-SLIM3 could transactivate a reporter gene expression in the myocardial cell line HL1, in the smooth muscle cell line A10, and in the embryonal kidney cell line 293.

SLIM3 is a functional androgen receptor co-activator: the reporter plasmid MMTV-LUC or proteasin-Luc was co-transfixed together with a constant amount of the androgen receptor-expression-plasmid in 293 cells. If the synthetic androgen receptor agonist R1881 were to be indicated, the reporter gene would be transactivated as a function of the ligands and receptors. A co-transfection of an increasing amount of SLIM3-expression plasmid resulted in an additional three-fold to five-fold super-induction of the reporter gene.

Similar results were obtained in CV1 cells. These data show that SLIM3 is a co-activator for nuclear receptor AR.

In comparison to other known co-activators, SLIM3 has an unusual specificity. SLIM3 regulates only the activity of two nuclear receptors, namely AR and ERβ. No other co-factor is known which carries out such a function. SLIM3 with nuclear receptors AR and ERβ thus can activate the corresponding translation in specific tissues or cells.

The entire disclosure of all applications, patents and publications, cited above and below, and of corresponding European Application No. 99 250 161.9, filed May 21, 1999, is hereby incorporated by reference.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method of identifying agents that regulate the transcriptional activating activity of human ERβ, comprising:
contacting a cell expressing human ERβ, and, human SLIM3, or biologically-active-derivatives thereof, with a test agent; and
determining whether said test agent regulates the transcriptional activating activity of human human ERβ

2. A method of claim 1, wherein said cell is a 293 cell or a yeast cell.

3. A method of claim 1, wherein said determining is measuring transcription of a gene activated by human ERβ.

4. A method of claim 1, wherein said human ERβis a chimeric protein comprising a GAL4 binding domain and SLIM3 is a chimeric protein comprising a GAL4 activator domain.

5. A method of claim 4, wherein said cell is a yeast cell comprising a β-galactosidase reporter gene.

6. A method of claim 5, where said yeast cell is Saccharomyces cerevisiae.

7. A method of claim 4, wherein said determining is measuring β-galactosidase activity.

8. A method of claim 5, wherein said determining is measuring B-galactosidase activity.

9. A method of claim 1, where said agent is an antagonist or an agonist.

10. A method of identifying agents that regulate the binding between SLIM3 and human ERβ, comprising:
contacting a sample comprising human SLIM3 and human ERβ, or biologically-active derivatives thereof, with a test agent; and
determining whether said test agent regulates the binding between said SLIM3 and said human ERβ.

11. A method of claim 9, where said SLIM3 is a chimeric protein comprising GST.

12. A composition comprising isolated human SLIM3 and isolated human ERβ.
